(19) United States
(12) Patent Application Publication
(10) Pub. No.: US 2025/0050567 A1
(43) Pub. Date: Feb. 13, 2025

(54) ONE-TIME INJECTION MOLDING METHOD FOR HUMAN BONE MODEL SAMPLES

(71) Applicant: Weidong LIU, Dayton, OH (US)

(72) Inventors: Weidong LIU, Dayton, OH (US); Yanbo LIU, Dayton, OH (US)

(21) Appl. No.: 18/232,624

(22) Filed: Aug. 10, 2023

(51) Int. Cl. B29C 45/76; A61B 6/00; A61B 34/10; B29C 33/38; C08J 3/22; C08J 9/00

(52) U.S. Cl. CPC B29C 45/7693; A61B 6/505; A61B 34/10; B29C 33/3835; C08J 3/22; C08J 9/0066; A61B 2034/105; B29K 2025/06; B29K 2105/04; B29L 2031/7532; C08J 2203/04; C08J 2325/06

(57) ABSTRACT

A one-time injection molding preparation method to imitate human bone samples is proposed, and a material database and a bone simulation model library are established that can effectively determine materials and the design process through the finite element method. In the polymer thermoplastic material, the filler with a higher density than the polymer material is first added, After granulation, it is mixed with a foaming agent or a filler with a lower density than the polymer thermoplastic materials. A human bone-like sample is obtained through one-time injection molding technology. This method saves experimental and labor costs and can be produced with traditional processing equipment. The sample is close to the density, hardness, and strength of real human bones, and is more consistent with the morphology and properties of real human bones than samples composed of multiple materials.

### TECHNICAL FIELD

The invention relates to the preparation of human bone model samples, in particular to a one-time injection molding preparation method.

### BACKGROUND TECHNIQUE

The human bone sample is a human-sized model product made of non-metallic materials, which is mainly used as a model for medical education and basic anatomy teaching. There are two main types of human bone model samples. One is the solid human bone morphology model made of polymer materials. It is only similar in shape but has no structural distinction between the cortical and spongy bone. It can only be used as a teaching display model. Another is the composite human bone samples made with polymer materials that have characteristics of human bone morphology: the cortical and the cancellous bone. Because this type of human bone-like samples is made of materials with different processing techniques, different materials have interface discontinuities. Therefore, the integration effect of the actual skeleton cannot be achieved.

In order to solve the above-mentioned various problems, the present invention proposes a one-time injection molding preparation method, which is granulated by adding a filler with a high density in the polymer thermoplastic material in advance, and then mixing a foaming agent or other material with lower density than that of the polymer thermoplastic material to achieve the raw materials for the one-time injection molding. Then, through one-time injection molding technology, a sample of human-like bones with bone cortex and cancellous shape and good simulation effect is obtained.

### CONTENTS OF INVENTION

The invention first obtains the mechanical performance test samples of various materials and different design formulas under different processing conditions, detects and obtains the physical parameters required for model design such as material density, hardness, and mechanical strength, and establishes a model material database.

Then, by scanning real human bone structures or CT data files, 3D morphological data is obtained, and a simulated bone model library of different bone shapes is established. According to the performance indicator requirements of bones in different parts, the design scheme of raw material composition, dosage and process conditions for preparing bionic bones by one-time injection molding is provided through finite element simulation analysis.

Based on the external dimensions of the human bone shape and the shrinkage and expansion coefficient of the material before and after molding, the injection mold may be designed.

Using various injection molding preparation sequences provided by finite element simulation analysis to repeatedly prepare bionic bones, test the performance indicators of bionic bones, continuously verify and optimize the simulation model, improve the accuracy of model and material composition, and finally obtain bone density, hardness, and strength close to real people in the bone model.

The concrete preparation process of the present invention is as follows:

A method for preparing a one-time injection mold imitating a human bone, comprising the following steps:
(1) Establish simulation material database: test the mechanical properties of various materials and their different design formulas under different processing conditions, obtain the physical parameters of material density, hardness, and mechanical strength required for simulation design, and establish a simulation material database
(2) Establish a simulated bone model library: by scanning real human bone structures or CT data files, obtain 3D morphological data, and establish a simulated bone model library of different bone shapes.
(3) Finite element method: based on the data of the simulated material database and the simulated bone model library, specifically the qualities of the target bone, obtain a design plan including raw material composition, dosage, and process conditions through computer finite element method analysis.
(4) Design injection molds: design molds according to the shape and size of human bones and the shrinkage and expansion coefficients of raw materials before and after molding.
(5) Preparation of basic masterbatch: following step (3), mix the polymer injection moldable material with the higher density filler “a”: at the temperature above the melting point of the polymer material, use a twin-screw granulator to create pellets, obtain a polymer mixture, and then dry to obtain a basic masterbatch; the density of the filler “a” is higher than that of the polymer injection moldable material.
(6) Preparation of raw material of the injection mold: mix the basic masterbatch with filler “b”, foaming agent and glass microsphere etc., to obtain the raw material of the injection: the density of filler “b” is lower than that of polymer injection moldable material.
(7) One-time injection molding: The injection molding raw material is injected into the mold in one go in the injection molding machine to obtain the human bone imitation sample.

The preparation method according to claim 1, wherein: the polymer injection material is selected from one or more mixtures of thermoplastics with similar or higher mechanical properties to real human bones: the thermoplastics include, but are not limited to: polyethylene, polypropylene, polystyrene, polymethyl methacrylate, polyvinyl chloride, polycarbonate, polyurethane, polytetrafluoroethylene, polyethylene terephthalate, polyoxymethylene, polyamide, polycarbonate, polyphenylene ether, polyethylene terephthalate, polysulfone, polyimide, polyphenylene sulfide, polycaprolactone, polylactic acid, polyether ether ketone.

The density of filler “a” is higher than that of polymer injectable materials, and it is selected from one or more inorganic materials such as inorganic salts and metal oxides, mainly to adjust the hardness and strength of bones, and especially to adjust the bone density of bionic bones and the hardness and strength of the cortex after molding. The filler “a” includes but is not limited to: calcium carbonate, clay, barium sulfate, silica, titanium oxide, calcium sulfate, mica, diatomaceous earth, kaolin, wollastonite, talc, aluminum hydroxide, asbestos, neodymium oxide, cerium fluorocarbon, rare earth salt of nitrate, rare earth salt of fatty acid, rare earth salt of stearate, rare earth salt of salicylic acid, rare earth salt of malic acid, rare earth salt of citrate and rare earth salt of tartaric acid, etc.

The density of filler “b” is lower than that of polymer injection mold materials, and it is selected from one or more solid and hollow inorganic fillers and sheet-like inorganic materials, mainly for adjusting the density of bones, which is mainly related to the adjustment of the cortical bone density. Filler “b” includes but is not limited to: hollow glass microspheres, expanded vermiculite, expanded perlite, etc.

As a preference, in step (6), glass microspheres perfused with red liquid can be optionally added to filler b and can be added to the basic masterbatch, which can produce the effect of bleeding when imitating anatomical human bones. The red liquid is an oily liquid added with a red pigment that appears red at room temperature, and the oily liquid is non-volatile under different melting and processing temperatures of polymer injection mold materials.

In step (6), the foaming agent is mainly to adjust the density of the bone, and the addition and dosage of the foaming agent are mainly related to the adjustment of the density of the cancellous bone.

In the injection molding machine barrel, along with the melting process of the polymer material, the foaming agent decomposes and forms bubble. The amount of the foaming agent is relatively small and is evenly dispersed into the injection molding raw material. Due to the processing pressure in the barrel, the size of the bubbles is small, and once entering the mold, the molten material first comes into contact with the mold whose temperature is much lower than the melting temperature, and the molten material quickly changes from melt to solid, forming the harder skin of the bone and then making it impossible for small-sized air bubbles to foam and form cortical bone. Because the molten material forms a temperature gradient in the bone cortex and the material away from the bone cortex, the tiny bubbles can expand and become larger in the mold with insufficient material under different temperature gradients, forming a morphological structure similar to the cancellous bone.

The foaming agent is selected from one material or a mixture of multiple materials whose foaming temperature is in the range of the melting temperature and the injection mold processing temperature of the polymer injection moldable material.

The foaming agent can be selected from azo compounds: foaming agent AC (azodicarbonamide, decomposition temperature 190° C.), DAB (azoaminobenzene, decomposition temperature 150° C.), etc.: can also be selected from sulfonyl hydrazine compounds: Benzenesulfonylhydrazide (foaming agent BSH, decomposition temperature about 100° C.), 4,4′-oxidized bisbenzenesulfonylhydrazide (foaming agent OBSH, decomposition temperature 195° C.), etc.: urea-based compounds: urea, p-formaldehyde sulfonylurea, etc.: organic foaming agent N-nitroso compound: N,N′-dinitrosopentamethylenetetramine (foaming agent H, BN, DPT, decomposition temperature 195-200° C.): Inorganic foaming agent: sodium bicarbonate (decomposition temperature about 300° C.), carbonate (such as ammonium bicarbonate, decomposition temperature 36-60° C.), nitrite (such as sodium nitrite-ammonium chloride mixture, decomposition temperature 175-320° C.), etc.

Further, foaming aids can also be added to the foaming agent. By adding foaming aids, the decomposition temperature of the foaming agent can be reduced to varying degrees, so as to adapt to the requirements of melting point and processing temperature of different polymer injection moldable materials. The foaming aid can be selected from: urea, urea derivatives, metal oxides, organic salts, borax and the like.

In step (5), the mass part of the polymer injection moldable material is 200, and the filler “a” is 30-120 parts. In the specific operation process, the selection of material types and the amount of additives can be determined according to physical parameters such as density, hardness, and mechanical strength of the target human bone.

In step (6), the mass part of the basic masterbatch is 200, the filler “b” is 0-20 parts, and the foaming agent is 0.5-3 parts. In the specific operation process, the selection of material types and the amount of additives can be determined according to physical parameters such as density, hardness, and mechanical strength of the target human bone.

The granulation process of step (5): for polymer injection molding materials with melting temperature above 180° C., the temperature of each zone of the twin screw granulator can be selected and set in the range of 180-320° C., after the temperature is reached, the mixed materials are blended and granulated to obtain the basic masterbatch.

The drying process of step (5): the drying temperature is 50° C.-80° C., and the drying duration is 0.5 h-4 h.

In step (7), for polymer injection molding materials with melting temperature above 180° C., the injection temperature of the injection molding machine can be selected from a range of 190° C.-310° C. After reaching a suitable temperature, the prepared injection raw materials are injected. The injection pressure can be selected from a range from 700-1300 kg/m².

In the preparation of injection molding of human bones, the traditional injection molding is to completely fill the entire cavity of the mold with the injection molding material, but in the one-time injection mold method of the present application, the amount of injection mold raw material is insufficient for injection molding, and the space required for foaming is left in the mold. The processing temperature range of traditional injection molding is wide and the injection molding speed is fast, while the injection molding conditions of this application require that the control range of processing temperature and injection molding speed is relatively narrow. If the control range of injection molding temperature and injection molding speed becomes larger during processing, the thickness and hole shape of cortical bone and cancellous bone will be affected. For example, if the temperature is too high during processing, the melt strength of the material will be reduced, making it easy to expand the bubbles when the material is injected into the mold, making it difficult to control the size of the hole within the design range. Excessive injection pressure will cause the product to have no cancellous bone structure, and the appearance is different from the real bone, while if the injection pressure is too low, artificial bone products the phenomenon of missing and melting joints

In the step (7), usually, the mold temperature is set at 40° C.-70° C., and the mold is opened after 50-100 seconds of injection molding to take out the imitation human bone sample.

The temperature range of the mold temperature is obtained through simulation design according to the material, processing conditions, test performance, bone shape and performance requirements, and different basic masterbatches and injection molding raw materials will have different mold temperature requirements.

By designing the mold temperature, the high-temperature thermoplastic material injected into the mold cools quickly after contact with the mold, and forms a bone-like surface similar to bone cortex because it has no time to foam.

The thickness of the cortical bone is related to the mold temperature, material formula and injection volume, and the thermoplastic material in the middle part maintained at a certain temperature will form spongy bone after foaming in the mold. The density of the spongy bone is related to the amount of foaming agent, injection temperature and time relevant. The middle part of the thermoplastic material that maintains a certain temperature forms cancellous bone after the foaming material is injected in the mold, and the density of cancellous bone is related to the amount of foaming agent, injection temperature and injection time.

If the mold temperature is higher than certain temperature range, the thickness of the cortical bone will be too thin, and the size of the holes in the cancellous bone will become larger; if the mold temperature is lower than the above temperature range, the thickness of the cortical bone will be too thick, and the size of the pores in the cancellous bone will become smaller. Too high or too low mold temperature will affect the degree of simulation of human bone samples.

The present invention has following beneficial effect:
(1) The present invention is an effective design that applies simulation technology to the preparation scheme of one-time injection molding artificial bone samples. Firstly, various materials that can be used, samples obtained under different processing conditions and performance test results are formed into a database. According to the bone shape and performance requirements, through finite element simulation analysis and design, the preparation plan required for one-time injection will be quickly and effectively provided with the accumulation and improvement of the material database and the simulated bone model library. It is a scientific and effective forward design method that solves the manpower and material resources spent on repeated designs and tests. The one-time injection molding products can be manufactured in traditional processing equipment.
(2) In the present invention, a filler with a high density is added to the polymer injection moldable material in advance to granulate, and after dyeing, a foaming agent and a filler lower than the density of the polymer injection moldable material are mixed to obtain a one-time Injection molding raw materials for preparing samples by one-time injection molding, and sample is manufactured by one-time injection molding. The addition of fillers higher than the density of the polymer injectable material is mainly to adjust the hardness and strength of the bone, especially to adjust the hardness and strength of the cortical bone in the bionic bone after molding. Foaming agents or fillers lower than the density of polymer injection moldable materials are mainly to adjust the density of bones. The addition and dosage of the foaming agent are mainly related to the adjustment of the density of the cancellous bone. The addition and dosage of fillers lower than the density of polymer injection moldable materials are mainly related to the adjustment of bone cortex density.

And if the red liquid imbedded hollow glass microspheres as a filler with foaming agent are added to the basic masterbatch, it is possible to prepare products that have the effect of bleeding when dissecting imitating human bones. And by optimizing and adjusting the injection molding process, such as injection temperature, injection rate, mold temperature, etc., a human bone-like sample with good simulation effect is prepared.
(3) The imitation human skeleton prepared by the present invention is functionally different from the common human skeleton models on the market. The imitation human skeleton of the present invention is designed according to real human skeleton data.
   It has been determined that the density of the human skeleton assay prepared by the present application is 0.82±0.05 g/cm³, the bending strength is 20±2 MPa, the bending deformation is 0.1±0.05 mm, the Shore hardness D is 81±1, and the molding shrinkage rate is 0.3-0.5%.

Using the same test method, the corresponding human bone density range is 0.75 g/cm³-0.84 g/cm³, the flexural strength range is 19.2 MPa-22.6 MPa, and the bending deformation range is 0.1 mm-0.2 mm (because the human bone adopts adult bone, the data is affected by age and physical condition, and the data has a certain deviation).

The above results show that the present application has physical performance parameters (such as density, hardness, strength, etc.) similar to real human bones and exhibits the outer cortex and internal cancellous of bones, which is more in line with the morphology and performance of real human bones, and can be used for teaching, preoperative simulation tests and medical device tests and practical operation training of doctors, etc., and has broad application prospects.

### SPECIFIC EXAMPLES

In order to make the purpose, technical solution and advantages of the present application clearer, the present application will be further described in detail below in conjunction with the embodiments. It should be understood that the specific embodiments described here are only used to explain the present application, and are not intended to limit the present application.

A one-time injection molding preparation method for human skeletal samples, including the following steps

Establish simulation material database: test the mechanical properties of various materials and their different design formulas under different processing conditions, obtain the physical parameters of material density, hardness, and mechanical strength required for simulation design, and establish a simulation material database:

Establish a simulated bone model library: by scanning real human bone structures or CT data files, obtain 3D morphological data, and establish a simulated bone model library of different bone shapes;

Finite element method: based on the data of the simulated material database and the simulated bone model library, specifically the qualities of the target bone, obtain a design plan including raw material composition, dosage, and process conditions through computer finite element method analysis:

Design injection molds: design molds according to the shape and size of human bones and the shrinkage and expansion coefficients of raw materials before and after molding:

Preparation of basic masterbatch: according to the design scheme obtained by finite element simulation analysis, mix the polymer injection moldable material with the higher density filler “a”: at the temperature above the melting point of the polymer material, use a twin-screw granulator to create pellets, obtain a polymer mixture, and then dry to obtain a basic masterbatch: the density of the filler “a” is higher than that of the polymer injection moldable material;

Preparation of raw material of the injection mold: mix the basic masterbatch with filler “b”, foaming agent and inorganic filler, such as glass microsphere etc., to obtain the raw material of the injection; the density of filler “b” is lower than that of polymer injection moldable material:

One-time injection molding: The injection molding raw material is injected into the mold in one go in the injection molding machine to obtain the human bone imitation sample.

### Example 1

Through scanning real human bone structures or CT data files, the design scheme of formula and process is obtained, and the mold design is carried out with the help of computer finite element simulation analysis.

Mix 200 parts of polystyrene, 5 parts of calcium carbonate and 35 parts of barium sulfate according to the mass fraction, in a mixer for 4 minutes, set the temperature of each zone of the twin screw granulator to 195° C. After the temperature was reached, the mixed materials were blended and granulated to obtain the base masterbatch, and the prepared base masterbatch was dried in the drying box at 60° C. for 1.5 hours.

The prepared base masterbatch and the foaming agent azodicarbonamide were prepared according to the mass ratio of 200:1, and mixed for 4 minutes to obtain injection molding raw materials. The temperature of each zone of the injection molding machine was set to 195° C., and after the temperature was reached, the injection molding raw materials were molded at one time, the injection pressure was 800 kg/m², the mold temperature was set to 40° C., and the sample of imitation leg bone was taken out after 65 seconds of injection molding.

It was determined by measuring samples that the average density of imitation leg bone sample was 0.82 g/cm³, the average bending strength was 20.2 MPa, and the average bending deformation was 0.10 mm. The average density of human bones (leg bones) obtained by the same test method was 0.83 g/cm³, the average bending strength was 20.3 MPa, and the average bending deformation was 0.11 mm.

By data comparison, it can be seen that the imitation leg bone sample prepared by the present embodiment has very similar physical performance parameters to the real leg bone. Through the appearance observation and comparison, the appearance shows the outer cortex and internal cancellous consistent with the real leg bone, and the degree of simulation is high, i.e., the human bone sample prepared in the present embodiment has a morphology and performance close to the real human bone.

### Example 2

Using the same leg bone as Example 1 as a comparison, change the mold temperature in the design scheme of Example 1, increase the mold temperature to 80° C. The other conditions were the same as Example 1: After observation, the thickness of the bone cortex of the imitation leg bone sample was thinner than that of the real bone, and the hole size of the cancellous bone of the imitation leg bone was larger than that of the real leg bone, and the simulation effect was not good. This is because the mold temperature was high, the cooling rate of the injection molding raw material after contact with the mold was small, and the degree of foaming of the foaming agent was excessive, so that the hole size of the finished product was larger than the real bone, which affected its appearance.

### Example 3

Using the same leg bone as Example 1 as a comparison, change the mold temperature in the design scheme of Example 1, reduce the mold temperature to 30° C. The other conditions were the same as Example 1: After observation, the thickness of the bone cortex of the humanoid bone sample was thicker than that of the real bone, and the hole size of the cancellous bone of the humanoid bone sample was smaller than that of the real bone, and the simulation effect was not good. This is because the mold temperature was low, the injection molding raw materials cooled faster after contact with the mold, and the foaming agent did not have time to foam, so that the hole size of the finished product was smaller than the real bone, which affected its appearance.

### Example 4

Using the same leg bone as Example 1 as a comparison, change the content of the filler barium sulfate in the raw material in the design scheme of Example 1, increase the amount of its addition to 65 parts, and other conditions are the same as Example 1: It was determined that the density of imitation leg bone sample was 0.94 g/cm³, the bending strength was 18.3 MPa, and the bending deformation was 0.08 mm. The density of human bones (leg bones) obtained by the same test method was 0.83 g/cm³, the bending strength was 20.3 MPa, and the bending deformation was 0.11 mm. After data comparison, the bone density range of humanoid bones was higher than that of real human bones, the average value of bending strength was lower than that of real human bones, and the amount of bending deformation was lower than that of real human bones.

### Example 5

Using the same leg bone as Example 1 as a comparison, the content of the filler barium sulfate in the raw material in the design scheme of Example 1 was changed, and the amount of addition was reduced to 5 parts, and other conditions were the same as in Example 1: It was determined that the density of imitation leg bone sample was 0.70 g/cm³, the bending strength was 20.5 MPa, and the bending deformation was 0.13 mm. The density of human bones (leg bones) obtained by the same test method was 0.83 g/cm³. the bending strength range was 20.3 MPa, and the bending deformation range was 0.11 mm. After data comparison, the bone density range of humanoid bones was lower than that of real human bones, the range of bending strength was higher than that of real human bones, and the amount of bending deformation was higher than that of real human bones.

### Example 6

Using the same leg bone as Example 1 as a comparison, the amount of foaming agent in the raw material in the design scheme of Example 1 was changed, and the amount of foaming agent azodicarbonamide was increased to 4 parts, and other conditions were the same as Example 1: It was determined that the density of imitation leg bone sample was 0.69 g/cm³, the bending strength was 19.6 MPa, and the bending deformation was 0.09 mm. The density of human bones (leg bones) obtained by the same test method was 0.83 g/cm³, the bending strength range was 20.3 MPa, and the bending deformation range was 0.11 mm. After data comparison, the bone density range of human-like bones was lower than that of real human bones, the range of bending strength was lower than that of real human bones, and the amount of bending deformation was lower than that of real human bones. From the appearance observation, the hole size of the human bone sample was larger than that of the real bone, and the appearance of the outer cortex and the internal cancellous was significantly different from the real bone. It was seen that the excessive amount of foaming agent affected the simulation degree of human bone samples.

### Example 7

Using the same leg bone as Example 1 as a comparison, the amount of foaming agent in the raw material in the design scheme of Example 1 was changed, the amount of foaming agent azodicarbonamide was reduced to 0.1 parts, and other conditions were the same as Example 1; It was determined that the density of imitation leg bone sample was 0.87 g/cm³, the bending strength was 20.6 MPa, and the bending deformation was 0.12 mm. The density of human bones (leg bones) obtained by the same test method was 0.83 g/cm³, the bending strength range was 20.3 MPa, and the bending deformation range was 0.11 mm. After data comparison, the bone density range of imitation human bones was higher than that of real human bones, the range of bending strength was higher than that of real human bones, and the amount of bending deformation was higher than that of real human bones. From the appearance observation, the hole size of the humanoid bone sample was smaller than that of the real bone, and the appearance of the outer cortex and the inner cancellous was different from the real bone. It was seen that too little foaming agent dosage affected the simulation degree of human bone samples.

### Example 8

Using the same leg bone as Example 1 as a comparison, change the amount of filler added to the injection molding raw material in the design scheme of Example 1, add 25 parts of hollow glass microspheres, and other conditions were the same as Example 1: It was determined that the density of imitation leg bone sample was 0.80 g/cm³, the bending strength was 19.2 MPa, and the bending deformation was 0.08 mm. The density of human bones (leg bones) obtained by the same test method was 0.83 g/cm³, the bending strength range was 20.3 MPa, and the bending deformation range was 0.11 mm. After data comparison, the bone density range of humanoid bones was lower than that of real human bones, the range of bending strength was lower than that of real human bones, and the amount of bending deformation was lower than that of real human bones, that is, too much filler with a density lower than that of polymer injection moldable materials was added, which affected the simulation degree of humanoid bone samples.

### Example 9

Using the same leg bone as Example 1 as a comparison, change the type of filler in the raw material in the design scheme of Example 1, replace the filler with a density higher than that of the polymer injection moldable material in the base masterbatch with a filler with a density lower than the polymer injection molding material, specifically, replace 5 parts of calcium carbonate and 35 parts of barium sulfate with 40 parts of hollow glass microspheres for granulation. The other conditions were the same as Example 1: It was determined that the density of imitation leg bone sample was 0.75 g/cm³, the bending strength was 19.4 MPa, and the bending deformation was 0.10 mm. The density of human bones (leg bones) obtained by the same test method was 0.83 g/cm³, the bending strength range was 20.3 MPa, and the bending deformation range was 0.11 mm. After data comparison, the bone density range of humanoid bones was smaller than that of real human bones, the flexural strength range was lower than that of real human bones, and the amount of bending deformation was lower than that of real human bones, which affected the simulation degree of humanoid bone samples.

### Example 10

Using the same leg bone as Example 1 as a comparison, change the formula of the injection molding raw material in the design scheme of Example 1, add a filler with a density higher than that of the polymer injection moldable material to the injection molding raw material, specifically, replacing the foaming agent with barium sulfate of the same quality. Other conditions were the same as Example 1: It was determined that the density of imitation leg bone sample was 0.89 g/cm³, the bending strength was 20.5 MPa, and the bending deformation was 0.12 mm. The density of human bones (leg bones) obtained by the same test method was 0.83 g/cm³, the bending strength range was 20.3 MPa, and the bending deformation range was 0.11 mm. After data comparison, the bone density range of human bones was higher than that of real human bones, but the flexural strength range was higher than that of real human bones, and the amount of bending deformation was higher than that of real human bones, and from the appearance point of view, it was difficult to distinguish between cancelling bone and bone cortex, which affected the simulation degree of human bone samples.

### Example 11

Using the same leg bone as Example 1 as a comparison, change the injection molding processing temperature in the design scheme of Example 1, adjust the temperature of each zone to 210° C. Other conditions were the same as Example 1: After appearance comparison analysis, the cancellous bone of the simulated product had large holes inside, and the bubble cells were uneven, which affected the simulation degree of human bone samples.

### Example 12

Using the same leg bone as Example 1 as a comparison, by changing the injection molding processing temperature in the design scheme of Example 1, the temperature of each zone was adjusted to 160° C. Other conditions were the same as Example 1: After appearance comparison analysis, the surface of the simulated product had welding marks, which affected the simulation degree of the human-like bone sample.

### Example 13

Using the same leg bone as Example 1 as a comparison, the injection pressure of the injection molding machine in the design scheme of Example 1 was changed from 800 kg/m² to 1500 kg/m², and other conditions were the same as Example 1: After appearance comparison analysis, when the pressure became larger, the simulated product did not have an obvious cancellous structure, and there was overflow burrs at the clamping part of the mold, which affected the simulation degree of the human bone sample.

### Example 14

Using the same leg bone as Example 1 as a comparison, the injection pressure of the injection molding machine in the design scheme of Example 1 was changed from 800 kg/m² to 600 kg/m², and other conditions were the same as Example 1: After appearance comparison analysis, the pressure became smaller, the product did not fully fill the mold, and the surface of the product presented the phenomenon of missing and melting joints, which affected the simulation degree of the human bone sample.

### Example 15

Using the same leg bone as Example 1 as a comparison, change the amount of injection molding raw materials per injection in the design scheme of Example 1. Other conditions were the same as Example 1; After data comparison, if the amount of injection molding raw materials was higher than the optimal amount of human bone products, there was a phenomenon similar to the larger injection pressure, i.e., simulated product did not have an obvious cancellous structure. When the injection molding amount was less than the optimal amount, there was a phenomenon similar to the smaller injection pressure, that is, the product was not completely filled with the mold, and the surface of the product presented the phenomenon of missing and melting joints, which affected the simulation degree of the human bone sample.

1. A one-time injection molding preparation method for imitating human bone samples, wherein, comprising the following steps:
(1) Establish simulation material database: test the mechanical properties of various materials and their different design formulas under different processing conditions, obtain the physical parameters of material density, hardness, and mechanical strength required for simulation design, and establish a simulation material database.
(2) Establish a simulated bone model library: by scanning real human bone structures or CT data files, obtain 3D morphological data, and establish a simulated bone model library of different bone shapes.
(3) Finite element method: based on the data of the simulated material database and the simulated bone model library, specifically the qualities of the target bone, obtain a design plan including raw material composition, dosage, and process conditions through computer finite element method analysis.
(4) Design injection molds: design molds according to the shape and size of human bones and the shrinkage and expansion coefficients of raw materials before and after molding.
(5) Preparation of basic masterbatch: following step (3), mix the polymer injection moldable material with the higher density filler “a”: at the temperature above the melting point of the polymer material, use a twin-screw granulator to create pellets, obtain a polymer mixture, and then dry to obtain a basic masterbatch; the density of the filler “a” is higher than that of the polymer injection moldable material.
(6) Preparation of raw material of the injection mold: mix the basic masterbatch with filler “b”, foaming agent and glass microsphere etc., to obtain the raw material of the injection: the density of filler “b” is lower than that of polymer injection moldable material.
(7) One-time injection molding: The injection molding raw material is injected into the mold in one go in the injection molding machine to obtain the human bone imitation sample.

2. The preparation method according to claim 1, wherein: the polymer injection material is selected from one or more mixtures of thermoplastics with similar or higher mechanical properties to real human bones: the thermoplastics include, but are not limited to: polyethylene, polypropylene, polystyrene, polymethyl methacrylate, polyvinyl chloride, polycarbonate, polyurethane, polytetrafluoroethylene, polyethylene terephthalate, polyoxymethylene, polyamide, polycarbonate, polyphenylene ether, polyethylene terephthalate, polysulfone, polyimide, polyphenylene sulfide, polycaprolactone, polylactic acid, polyether ether ketone.

3. The preparation method according to claim 1, wherein: the density of filler “a” is higher than that of the polymer injection material, and is selected from one or more of inorganic salts and metal oxides.

4. The preparation method according to claim 1, wherein: the density of the filler “b” is lower than that of the polymer injection material, and is selected from one or more of solid, hollow inorganic fillers and sheet-like inorganic materials.

5. The preparation method according to claim 1, wherein: in step (6), as glass microspheres perfused with red liquid are added, the red liquid is an oily liquid with red pigment, which appears red at room temperature, The oily liquid is non-volatile under the melting and processing temperature conditions of different polymer injection materials.

6. The preparation method according to claim 1, wherein: in step (6), the foaming agent is selected from one or more of the following materials: azo compounds: azodicarbonamide, azoaminobenzene; sulfonylhydrazide compounds: benzenesulfonyl hydrazide, 4,4′-oxidized bisbenzenesulfonyl hydrazide; urea-based compounds: urea, p-methylsulfonylurea; organic foaming agent: N,N′-dinitrosopentamethyltetramine; inorganic foaming agent: sodium bicarbonate, ammonium bicarbonate, sodium nitrite-ammonium chloride mixture.

7. The preparation method according to claim 1, wherein: in step (5), the mass part of the polymer injection moldable material is 200, and the filler “a” is 30-120 parts.

8. The preparation method according to claim 1, wherein: in the step (6), the mass part of the basic masterbatch is 100, the filler “b” is 0-20 parts, and the foaming agent is 0.5-3 parts.

9. The preparation method according to claim 1, wherein: in step (7), the injection temperature of the injection molding machine can be selected from a range of 190° C. to 310° C., and the injection pressure can be selected from the range of 700-1300 kg/m².

10. The preparation method according to claim 1, wherein: in step (7), the mold temperature is set at 40° C.-70° C., and the mold is opened after 50-100 seconds of injection molding to take out the human bone imitation sample.
